(19) United States
(12) Patent Application Publication
(10) Pub. No.: US 2022/0233336 A1
(43) Pub. Date: Jul. 28, 2022

(54) Pre-operative Planning and Instrument Optimization Method and System for Primary Knee Replacement Procedures

(71) Applicant: Ashesh Shah, Newtown Square, PA (US)

(72) Inventor: Ashesh Shah, Newtown Square, PA (US)

(21) Appl. No.: 16/566,869

(22) Filed: Sep. 10, 2019

(51) Int. Cl. A61F 2/46; A61F 2/38; G16H 20/40

(52) U.S. Cl. CPC A61F 2/4657; A61F 2/389; A61F 2002/4658; A61F 2002/4633; G16H 20/40

(57) ABSTRACT

A method and system for use by a health-care provider for pre-operatively determining the optimal size and shape of a replacement knee prosthesis for a patient. The maximum medial- lateral width of the patient's proximal tibia is measured and used to determine the optimal-size tibial component selected from a set of tibial components of varying size using data correlating the medial-lateral width with an optimal tibial component size. The size of the tibial component is then used to determine the optimal size of the femoral component selected from a set of tibial components of varying size using data correlating the tibial size with an optimal femoral component size. The size of the tibial and femoral components are then used to determine the optimal size of the tibial liner selected from a set of tibial liners of varying size using data correlating the tibial size with an optimal tibial liner size. The system includes a personal communications device running an operative mobile device application program, which can automatically measure the medial-lateral width and correlate the medial-lateral width with an optimal tibial component size, optimal femoral component size, and optimal tibial liner size.

### FIELD OF THE INVENTION

The present invention relates to a method and apparatus for pre-operatively determining the optimal size and shape of a replacement knee prosthesis.

### BACKGROUND OF THE INVENTION

Total knee replacement surgery involves resecting the distal femur and proximal tibia, and then installing a replacement femoral component and tibial component. The size, and to some extent the shape, of the replacement components will vary depending on the patient's size and physique. It is common for the surgeon to make the size and shape determination during surgery after the patient's femur and tibia are exposed and resected. The surgeon then selects the best prosthesis from a kit having multiple prostheses components of varying sizes and shapes. The remaining prostheses that were not selected are then returned for re-sterilization and potential use in another patient.

It is obviously expensive and wasteful to provide surgeons with multiple prostheses when only one of each component will be selected. Therefore, it is desirable to provide a method of pre-operatively determining the prostheses components that best fit a given patient so that a reduced number of component sizes and/or shapes, or only a single component size, need be provided pre-operatively to the surgeon.

Known methods of pre-operatively sizing a patient to determine the best prosthesis size include x-ray imaging, computerized axial tomography (CAT scanning), and magnetic resonance imaging (MRI). However, such imaging is very expensive, on the order of hundreds to thousands of dollars per scan. Furthermore, sizing can only be done after the imaging results are made available, and hence often requires multiple visits, which also adds to the total cost of care. Therefore, it would be desirable to provide a simple and inexpensive method of pre-operatively determining the optimal prosthesis size and/or shape for any given patient.

### SUMMARY OF THE INVENTION

The present invention provides a method and system for use by a health-care provider (“HCP”) for pre-operatively determining the optimal size and/or shape of a replacement knee prosthesis for a patient. In a preferred embodiment, any clothing items are removed to visually expose the knee. The knee is then bent from full extension to a position preferably less than about 90 degrees of flexion. The maximum medial-lateral width of the patient's proximal tibia is measured. Then, the measured width is used to determine the optimal-size tibial component selected from a set of tibial components of varying size using data correlating the medial-lateral width with an optimal tibial component size. The size of the tibial component is used to determine the optimal size of the femoral component selected from a set of tibial components of varying size using data correlating the tibial size with an optimal femoral component size. The size of the tibial and femoral components is used to determine the optimal size of the tibial liner selected from a set of tibial liners of varying size using data correlating the tibial size with an optimal tibial liner size.

In one preferred embodiment, the measuring step initially comprises providing a personal communications device (“PCD”) running an operable augmented reality mobile device application program (“AR app”). The knee is then imaged with the PCD and the AR app. Preferably, the PCD automatically correlates the medial-lateral width with a data lookup table to determine the optimal tibial component size.

In another embodiment of the invention, the number of components in an orthopedic surgical instrument case is reduced by transmitting the data identifying the optimal tibial, femoral and liner component sizes to an orthopedic surgical instrument case manufacturer. The manufacturer then provides trial implants and cutting guides only for the optimal tibial, femoral and liner component sizes. Optionally, the method could include the step of providing trial implants that are one size larger and one size smaller than the optimal tibial, femoral and liner components sizes.

In another embodiment, the measuring width of the proximal tibia is measured by applying a plurality of visually detectable markers to the exterior of the proximal tibia. A personal communications device running a mobile device application program. Then, the distance between markers is measured using the PCD and the mobile device application program.

In yet another preferred embodiment, an additional marker distance is measured between at least two external markers on a different portion of the patient's body. The measured width and the marker distance are then used to determine the optimal size tibial component selected from a set of tibial components of varying size using data correlating the measured distance and marker distance with an optimal tibial component size. The additional marker distance may be determined by measuring at least one of the anterior-posterior foot size, medial-lateral foot size, anterior-posterior proximal tibial size, and height of the patient.

### BRIEF DESCRIPTION OF THE DRAWINGS

FIG. 1 is a lateral view of the lower portion of a human leg illustrating dimensions used in accordance with a preferred embodiment of the invention; and ,

FIG. 2 is an anterior view of the lower portion of a human leg illustrating dimensions used in accordance with a preferred embodiment of the invention.

### DETAILED DESCRIPTION OF THE PREFERRED EMBODIMENT(S)

The invention allows the user, such as a surgeon or other health-care provider (“HCP”), to determine the size and/or shape of the patient's femur and tibia, and thereby determine the optimal size and/or shape of replacement components that will fit the patient. It is assumed that the HCP is aware of the sizing measurements of the components they use, specifically the medial-lateral and anterior-posterior dimensions of the femoral and tibial components.

AKS Development Approach—In one preferred embodiment, the present invention uses the new apple ARKit to eliminate expensive imaging requirements or multiple visits. Instead, the invention can be used to pre-operatively and immediately size a patient's knee in the examination room in less than a few minutes by measuring external markers on the patient.

In accordance with a preferred method, a mobile device application program (“app”) is provided that utilizes an augmented reality app (“ARKit”). One such app is MeasureKit. The app allows the HCP to measure the distance between two points in space. In another embodiment, the app also allows the HCP to map the shape of the bone using the built-in gyro and account for curvatures when building sizing calculations to optimize bone coverage.

Next, the HCP flexes the patient's knee to a maximum of 90 degrees. Then, the HCP measures the farthest medial-lateral points on the proximal tibia of the patient (“Tib Size”). See sketch A. Then, either manually or via a mobile app, the HCP determines the optimal fit, i.e., size and/or shape, of the tibial component which matches the patient's Tib Size (the “TibComponent”). Once the TibComponent is determined, either manually or via a mobile app, the HCP determines the universe of femoral components that match the TibComponent (“FemComponent(s)”). Based on the FemComponent(s) and the TibComponent, the HCP, either manually or via a mobile app, can determine the list of compatible tibial liners that will match these components (“LinerComponents”)

After the HCP has determined the best TibComponent, FemComponent(s), and LinerComponents for the patient, this information is conveyed to the manufacturer. Once the manufacturer receives this information, it can construct an instrument case for the patient's surgery that is optimized based on this sizing information. This optimization includes: providing trial implants only for the optimized TibComponent, FemComponent(s) and LinerComponents; and, providing cutting guides only for optimized TibComponent, FemComponent(s) and LinerComponents. The manufacturer will deliver this optimized instrument tray to the surgical site in time for the patient's surgery.

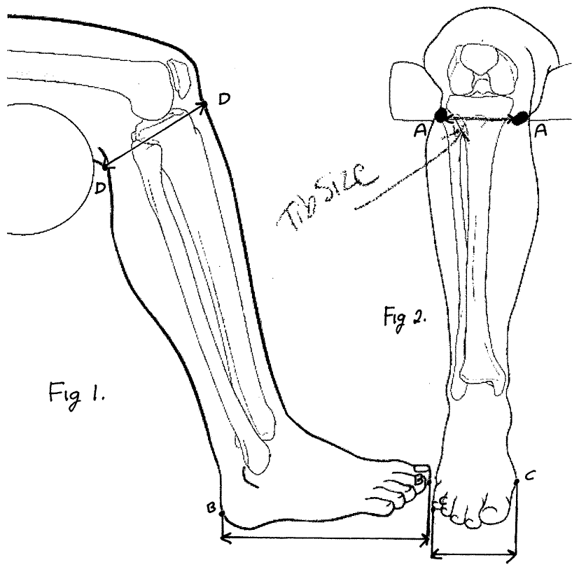

In another preferred embodiment of the invention, external markers on a different portion of the patient's body is measured and used to correlate the optimal size of the tibial component, femoral component and/or tibial liner. For example, based on accumulated data, there is a correlation between the following measurements and the optimal prostheses components: (1) the anterior-posterior foot size as measured from the heel to the big, markers B-B FIG. 1; (2) medial-lateral foot size as measured from the outer toe to the bunion plane, markers C-C of FIG. 2; the anterior-posterior size of the proximal tibia, markers D-D of FIG. 1; and/or, (4) height of the patient.

1. A method for use by a health-care provider (“HCP”) for pre-operatively determining the optimal size and/or shape of a replacement knee prosthesis for a patient, comprising the steps of:
a. measuring the maximum medial-lateral width of the patient's proximal tibia; and,
b. using the measured width to determine the optimal size tibial component selected from a set of tibial components of varying size using data correlating the medial-lateral width with an optimal tibial component size.

2. The method recited in claim 1, including the steps of:
c. using the size of the tibial component to determine the optimal size of the femoral component selected from a set of tibial components of varying size using data correlating the tibial size with an optimal femoral component size.

3. The method recited in claim 2, including the steps of:
d. using the size of the tibial and femoral components to determine the optimal size of the tibial liner selected from a set of tibial liners of varying size using data correlating the tibial size with an optimal tibial liner size.

4. The method recited in claim 1, wherein the measuring step comprises bending the knee from full extension to a position less than 90 degrees of flexion.

5. The method recited in claim 1, wherein the measuring step comprises removing any clothing items to visually expose the knee.

6. The method recited in claim 1, wherein the measuring step comprises:
a. providing a personal communications device (“PCD”) running an operable augmented reality mobile device application program (“AR app”); and,
b. imaging the knee with the PCD and the AR app.

7. The method recited in claim 6, including the step of (c) using the PCD to automatically correlating the medial-lateral width with a data lookup table to determine the optimal tibial component size.

8. The method recited in claim 3, including the step of reducing the number of components in an orthopedic surgical instrument case by:
a. transmitting the data identifying the optimal tibial, femoral and liner component sizes to an orthopedic surgical instrument case manufacturer;
b. providing trial implants only for the optimal tibial, femoral and liner component sizes; and,
c. providing cutting guides sized only for the optimal tibial, femoral and liner component sizes.

9. The method recited in claim 8, including the step of providing trial implants that are one size larger and one size smaller than the optimal tibial, femoral and liner components sizes.

10. The method recited in claim 1, wherein the measuring step comprises:
a. applying a plurality of visually detectable markers to the exterior of the proximal tibia; and
b. providing a personal communications device (“PCD”) running a mobile device application program; and,
c. measuring the distance between markers using the PCD and the mobile device application program.

11. The method recited in claim 1, including the step of:
c. measuring an additional marker distance between at least two external markers on a different portion of the patient's body; and,
d. using the measured width and the marker distance to determine the optimal size tibial component selected from a set of tibial components of varying size using data correlating the measured distance and marker distance with an optimal tibial component size.

12. The method recited in claim 11, wherein the additional marker distance is determined by measuring at least one of the anterior-posterior foot size, medial-lateral foot size, anterior-posterior proximal tibial size, and height of the patient.

13. A method for use by a health-care provider (“HCP”) for pre-operatively determining the optimal size and shape of a replacement knee prosthesis for a patient, comprising the steps of:
a. measuring the distance between least two external markers on a portion of the patient's body;
b. using the measured distance to determine the optimal size tibial component selected from a set of tibial components of varying size using data correlating the medial-lateral width with an optimal tibial component size;
c. using the size of the tibial component to determine the optimal size of the femoral component selected from a set of tibial components of varying size using data correlating the tibial size with an optimal femoral component size; and,
d. using the size of the tibial and femoral components to determine the optimal size of the tibial liner selected from a set of tibial liners of varying size using data correlating the tibial size with an optimal tibial liner size.

14. A system for use by a health-care provider for pre-operatively determining the optimal size and shape of a replacement knee prosthesis for a patient, comprising a personal communications device running an operative mobile device application program, which can measure the distance between two known patient markers, and correlate said distance with an prosthesis size for the patient.
